(19) United States
(12) Patent Application Publication
(10) Pub. No.: US 2010/0136111 A1
(43) Pub. Date: Jun. 3, 2010

(54) PHARMACEUTICAL COMPOSITIONS OF DICLOFENAC AND MISOPROSTOL

(76) Inventors: Ramakant Gundu, Maharashira (IN); Girish Kumar Jain, Delhi (IN); Murali Narayanan, Tamil Nadu (IN); Rahul Dabre, Maharashtra (IN); Mandar Kodgule, Maharashtra (IN)

Correspondence Address: BIO INTELLECTUAL PROPERTY SERVICES (BIO IPS) LLC, 8509 KERNON CT., LORTON, VA, 22079, US

(21) Appl. No.: 12/530,680

(22) PCT Filed: Mar. 25, 2008
(86) PCT No.: PCT/IB2008/051090

§ 371 Date: Jan. 27, 2010

(30) Foreign Application Priority Data
Mar. 30, 2007 (IN) 605/MUM/2007
Mar. 30, 2007 (IN) 606/MUM/2007
Mar. 30, 2007 (IN) 611/MUM/2007
Mar. 30, 2007 (IN) 612/MUM/2007
Mar. 30, 2007 (IN) 617/MUM/2007

(51) Int. Cl. A61K 31/25; A61K 9/20; A61K 9/28; A61K 9/30; A61K 9/32; A61K 9/36; A61P 29/00; A61P 1/04

(52) U.S. Cl. 424/465; 424/474; 424/475; 424/480; 424/482; 514/530

(57) ABSTRACT

The present invention relates to pharmaceutical compositions of diclofenac or pharmaceutically acceptable salts thereof and misoprostol or pharmaceutically acceptable salts thereof. The invention also relates to processes for the preparations of such compositions.

### FIELD OF THE INVENTION

The present invention relates to pharmaceutical compositions of diclofenac or pharmaceutically acceptable salts thereof and misoprostol or pharmaceutically acceptable salts thereof. The invention also relates to processes for the preparations of such compositions.

### BACKGROUND OF THE INVENTION

Diclofenac sodium is a phenyl acetic acid derivative, which is chemically 2-[(2,6-dichlorophenyl)amino]benzeneacetic acid, monosodium salt.

Misoprostol is chemically (±) methyl 11a,16-dihydroxy-16-methyl-9-oxoprost-13E-en-1-oate.

Arthrotec® (diclofenac sodium/misoprostol) is a combination product containing diclofenac sodium, a non-steroidal anti-inflammatory drug (NSAID) with analgesic properties, and misoprostol, a gastrointestinal (GI) mucosal protective prostaglandin E1 analog. Arthrotec oral tablets are white to off-white, round, biconvex and approximately 11 mm in diameter. Each tablet consists of an enteric-coated core containing 50 mg or 75 mg diclofenac sodium surrounded by an outer mantle containing 200 mcg misoprostol.

U.S. Pat. Nos. 5,601,843 and 5,698,225 disclose a tablet having a core of a NSAID selected from diclofenac and piroxicam. The core is surrounded by a mantle coating of a prostaglandin such as misoprostol, wherein an intermediate coating can be present between the NSAID core and the prostaglandin mantle coating.

U.S. Pat. No. 5,015,481 describes compositions that include an admixture of an NSAID selected from diclofenac and piroxicam, a prostaglandin such as misoprostol, and a stabilizer, preferably hydroxypropyl methylcellulose.

U.S. Pat. No. 6,740,340 discloses a pharmaceutical tablet that incorporates two smaller tablets, one of which includes an NSAID and the other one includes misoprostol, preferably in the form of dispersion in hydroxypropyl methylcellulose.

U.S. Pat. Nos. 6,511,680 and 6,319,519 disclose a dosage form that includes an NSAID in coated pellets and misoprostol is located outside the pellets in the form of a solid dispersion in hydroxypropyl methylcellulose or polyvinylpyrrolidone.

U.S. Pat. Nos. 6,183,779 and 6,287,600 disclose a dosage form that includes an NSAID present in enteric coated granules or particles and misoprostol is located outside the pellets in the form of a solid dispersion in hydroxypropyl methylcellulose or polyvinylpyrrolidone. U.S. Pat. Nos. 6,387,410; 6,514,525; 6,537,582 and 6,787,155 disclose a similar dosage form that includes the NSAID containing pellets in a delayed release formulation.

U.S. Pat. No. 6,656,503 discloses a tablet dosage form that includes a core and a film coating, wherein the core includes an NSAID and the film coating includes a polymer and misoprostol.

U.S. Pat. No. 5,232,704 discloses a capsule dosage form containing one layer that includes a drug release layer containing misoprostol and the other a buoyant or floating layer.

U.S. Application No. 2005163847 discloses a solid dosage form that includes a first portion comprising NSAID; and a coating containing an antiulcerative compound, said coating at least partially surrounding the NSAID portion.

U.S. Application No. 20040185100 discloses a dual release dosage form that includes an extended release NSAID and an immediate release stabilized prostaglandin.

U.S. Application No. 2005031690 discloses a dosage form that includes a plurality of zones, at least one of which includes an NSAID and another of which includes a solid dispersion of a prostaglandin type compound in hydroxypropyl methylcellulose.

European Patent Application No. 1020182 discloses a two-layer tablet having an NSAID and misoprostol located in separate layers. The misoprostol can be present in the form of a solid dispersion in hydroxypropyl methylcellulose.

European Patent Application No. EP1216030 discloses a dosage form including a mixture of a delay release formulation of NSAID and a mixture containing a prostaglandin and one or more excipients.

NSAIDs present great therapeutic benefit in the treatment of inflammatory conditions such as arthritis, but have an ulcerogenic effect in the upper gastrointestinal tract, which can seriously limit their usefulness, especially for chronic treatment. Certain prostaglandin type compounds, especially prostaglandin E1 derivatives and more particularly, misoprostol has been found to mitigate or provide protection against such ulcerogenic effects when co-administered with an NSAID.

Chemical degradation of certain prostaglandin type compounds, particularly prostaglandin E1 derivatives such as misoprostol, is accelerated in the presence of water, and the primary pathway of degradation is believed to be dehydration to the corresponding prostaglandin A derivative. The problem of chemical instability becomes more acute when the prostaglandin type compound is co-formulated with certain NSAIDs such as diclofenac or piroxicam.

The present invention addresses and overcomes these commonly encountered problems.

### SUMMARY OF THE INVENTION

In one general aspect there is provided a tablet in a tablet dosage form of diclofenac and misoprostol. The dosage form includes an inner tablet that includes misoprostol or a salt thereof and optionally other pharmaceutically acceptable excipients and an outer tablet that includes coated beads of diclofenac or a salt thereof and optionally other pharmaceutically acceptable excipients.

The pharmaceutically acceptable excipients may include one or more of fillers, binders, lubricants, glidants, disintegrants, and the like.

Embodiments of the dosage form may include one or more of the following features. For example, the dosage form may exhibit a dissolution profile such that within first 2 hours less than 2% of diclofenac or a salt thereof is released when the release rate is measured in Apparatus 2 (USP, Dissolution, paddle, 50 rpm) using 900 ml of 0.1N HCl at 37° C.±0.5° C. and within first 30 minutes more than 80% of diclofenac or a salt thereof is released, when the release rate is measured in Apparatus 2 (USP, Dissolution, paddle, 50 rpm) using 1000 ml of pH 6.8 phosphate buffer at 37° C.±0.5° C.

The dosage form may include a coating. The tablet may be coated with one or more enteric polymers or pharmaceutically acceptable seal coat polymers.

In another general aspect there is provided a dosage form. The dosage form includes coated minitablets of diclofenac or a salt thereof optionally, with other pharmaceutically acceptable excipients and beads of misoprostol or a salt thereof optionally, with other pharmaceutically acceptable excipients.

Embodiments of the dosage form may include one or more of the following features. For example, the dosage form may exhibit a dissolution profile such that within first 2 hours less than 2% of diclofenac or a salt thereof is released when the release rate is measured in Apparatus 2 (USP, Dissolution, paddle, 50 rpm) using 900 ml of 0.1N HCl at 37° C.±0.5° C. and within first 30 minutes more than 80% of diclofenac or a salt thereof is released, when the release rate is measured in Apparatus 2 (USP, Dissolution, paddle, 50 rpm) using 1000 ml of pH 6.8 phosphate buffer at 37° C.±0.5° C.

The pharmaceutically acceptable excipients may include one or more of fillers, binders, lubricants, glidants, disintegrants, and the like.

In another general aspect there is provided a pillow tablet dosage form of diclofenac and misoprostol. The dosage form includes an inner-pillowed tablet that includes misoprostol or a salt thereof and optionally other pharmaceutically acceptable excipients and an outer tablet that includes coated beads of diclofenac or a salt thereof and optionally other pharmaceutically acceptable excipients.

The term ‘inner-pillowed tablet’ as used herein refers to an inner tablet of misoprostol tilted on one side of the diclofenac tablet surface, such that it looks like a pillow at the center.

Embodiments of the dosage form may include one or more of the following features. For example, the dosage form may exhibit a dissolution profile such that within first 2 hours less than 2% of diclofenac or a salt thereof is released when the release rate is measured in Apparatus 2 (USP, Dissolution, paddle, 50 rpm) using 900 ml of 0.1N HCl at 37° C.±0.5° C. and within first 30 minutes more than 80% of diclofenac or a salt thereof is released, when the release rate is measured in Apparatus 2 (USP, Dissolution, paddle, 50 rpm) using 1000 ml of pH 6.8 phosphate buffer at 37° C.±0.5° C.

The pharmaceutically acceptable excipients may include one or more of fillers, binders, lubricants, glidants, disintegrants, and the like.

In another general aspect there is provided an inlay tablet dosage form of diclofenac and misoprostol. The dosage form includes an inner inlayed tablet that includes misoprostol or a salt thereof and optionally other pharmaceutically acceptable excipients and an outer tablet that includes coated beads of diclofenac or a salt thereof and optionally other pharmaceutically acceptable excipients.

The term ‘inlayed tablet’ as used herein refers to a type of a layered tablet in which instead of the core tablet being completely surrounded by a coating, the top surface is completely exposed.

Embodiments of the dosage form may include one or more of the following features. For example, the dosage form may exhibit a dissolution profile such that within first 2 hours less than 2% of diclofenac or salt thereof is released when the release rate is measured in Apparatus 2 (USP, Dissolution, paddle, 50 rpm) using 900 ml of 0.1N HCl at 37° C.±0.5° C. and within first 30 minutes more than 80% of diclofenac or salt thereof is released, when the release rate is measured in Apparatus 2 (USP, Dissolution, paddle, 50 rpm) using 1000 ml of pH 6.8 phosphate buffer at 37° C.±0.5° C.

The pharmaceutically acceptable excipients may include one or more of fillers, binders, lubricants, glidants, disintegrants, and the like.

In another general aspect there is provided a dosage form that includes coated beads of diclofenac or a salt thereof optionally, with pharmaceutically acceptable excipients and a coating that includes misoprostol or a salt thereof optionally, with pharmaceutical acceptable excipients, characterized in that the misoprostol coating covers not more than 90% of the coated beads of diclofenac or a salt thereof.

Embodiments of the dosage form may include one or more of the following features. For example, the dosage form may exhibit a dissolution profile such that within first 2 hours less than 2% of diclofenac or salt thereof is released when the release rate is measured in Apparatus 2 (USP, Dissolution, paddle, 50 rpm) using 1000 ml of 0.1N HCl at 37° C.±0.5° C. and within first 30 minutes more than 80% of diclofenac or salt thereof is released, when the release rate is measured in Apparatus 2 (USP, Dissolution, paddle, 50 rpm) using 1000 ml of pH 6.8 phosphate buffer at 37° C.±0.5° C.

The pharmaceutically acceptable excipients may include one or more of fillers, binders, lubricants, glidants, disintegrants, and the like.

The details of one or more embodiments of the inventions are set forth in the description below. Other features, objects and advantages of the inventions will be apparent from the description and claims.

### DESCRIPTION OF THE DRAWINGS

FIG. 1 shows a diclofenac-misoprostol tablet dosage form of G.D. Searle (Arthrotec®)

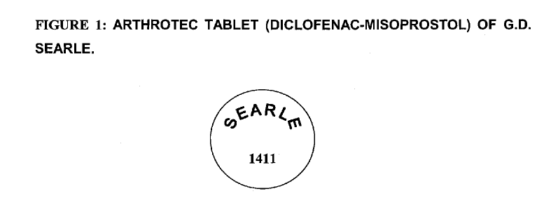

FIG. 2 shows some of the examples of diclofenac-misoprostol tablet in a tablet dosage form of the present invention.

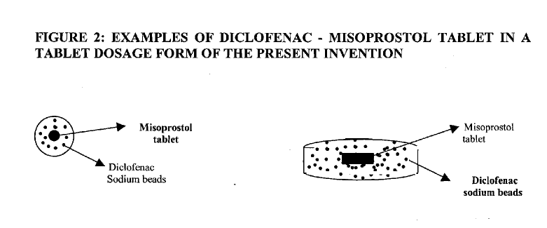

FIG. 3 shows some of the examples of diclofenac-misoprostol pillow tablet dosage form of the present invention.

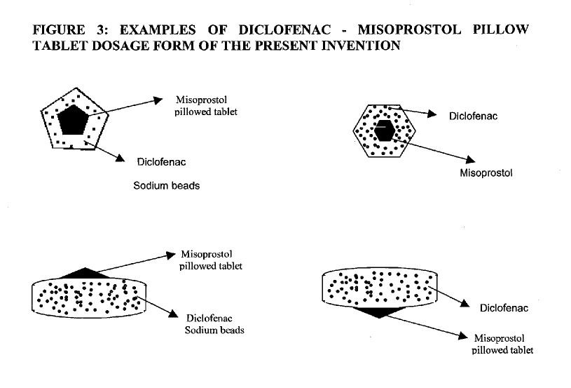

FIG. 4 shows some of the examples of diclofenac-misoprostol inlay tablet dosage form of the present invention.

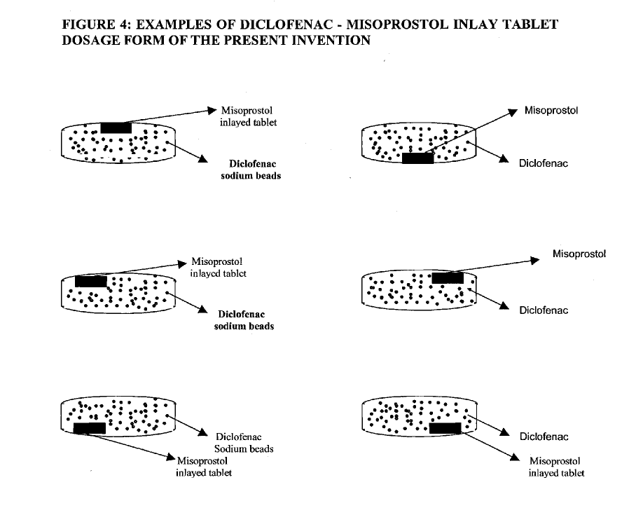

### DETAILED DESCRIPTION OF THE INVENTION

The inventors have now discovered that when misoprostol is not in a direct contact with diclofenac and outer environmental conditions, misoprostol is not degraded and a stable formulation can be prepared. According to one embodiment, when misoprostol is present as an inner tablet either inlayed or pillowed within the outer diclofenac tablet, wherein the diclofenac is present in form of coated beads, misoprostol is not exposed to both diclofenac and outer environmental conditions; hence it is prevented from degradation.

In yet another embodiment, when diclofenac is present in the form of coated minitablets and misoprostol is present in the form of beads, misoprostol is not exposed to diclofenac due to the presence of an intermediate seal coat and enteric coat between diclofenac and misoprostol; hence it is prevented from degradation. Further, the inventors have discovered that even when misoprostol coats 90% of the enteric coated beads of diclofenac or a salt thereof, misoprostol is not in direct contact with diclofenac due to the presence of an intermediate seal coat and enteric coat and hence it is prevented from degradation.

In all the above-mentioned embodiments, the exposure of misoprostol to diclofenac and outer environmental conditions is prevented, thus resulting in the prevention of degradation of misoprostol and a stable formulation.

In one embodiment, a tablet in a tablet dosage form of diclofenac and misoprostol may be prepared by compressing a blend of enteric-coated beads of diclofenac or a salt thereof with a misoprostol tablet along with other pharmaceutically acceptable excipients such that inner tablet is of misoprostol or a salt thereof and outer tablet is of diclofenac or a salt thereof along with other pharmaceutically acceptable excipients.

In another embodiment, diclofenac and misoprostol dosage form may be prepared by compressing a blend of enteric-coated beads of diclofenac or a salt thereof with a misoprostol tablet along with other pharmaceutically acceptable excipients into a pillow tablet dosage form in such a way that misoprostol tablet is tilted on one side of the diclofenac tablet surface, such that it looks like a pillow at the center.

In still another embodiment, diclofenac and misoprostol dosage form may be prepared by compressing a blend of enteric-coated beads of diclofenac or a salt thereof with a misoprostol tablet along with other pharmaceutically acceptable excipients into an inlay tablet dosage form in such a way that misoprostol tablet is inlayed on one side of the diclofenac tablet surface.

In still another embodiment, diclofenac and misoprostol dosage form may be prepared by mixing diclofenac loaded enteric-coated beads with other pharmaceutically acceptable excipients. The diclofenac loaded enteric coated beads blend may be divided into two parts in such a way that first part contains 90% of the enteric-coated beads of diclofenac and other second part contains the remaining 10% of the enteric-coated beads of diclofenac. The first part containing 90% of the enteric-coated beads of diclofenac may be mixed with misoprostol-hypromellose dispersion. The enteric-coated diclofenac beads coated with misoprostol-hypromellose dispersion and the second part containing the remaining 10% of the enteric-coated beads of diclofenac may be processed into a suitable dosage form.

The coated beads of diclofenac or a salt thereof may be prepared by coating inert spherical beads with a suspension of diclofenac or a salt thereof; overcoating the diclofenac loaded beads with a pharmaceutically acceptable seal coat polymer; enteric-coating the seal coated diclofenac beads with a pharmaceutically acceptable enteric coat polymer; mixing the enteric-coated beads of diclofenac or a salt thereof with polyethylene glycol and optionally with other pharmaceutically acceptable excipients.

During compression, pressure exerted on beads results in cracking of the beads. The presence of polyethylene glycol in the enteric-coated diclofenac beads provides cushioning effect to beads and avoids cracking under compression pressure.

Polyethylene glycol may include one or more of PEG 2000, PEG 4000, PEG 3350, PEG 6000, PEG 8000, and the like.

The inert spherical beads may be made up of one or more of saccharides or derivatives thereof such as polysaccharides, sugars such as mannitol, sorbitol, lactose, sucrose, maltodextrin, starch and its derivatives such as maize starch, rice starch, celluloses such as microcrystalline cellulose, sodium carboxymethylcellulose, and the like.

The suspension of diclofenac or a salt thereof may be made up of diclofenac or a salt thereof along with one or more hydrophilic polymers, water and pharmaceutically acceptable excipients.

Suitable hydrophilic polymers may include one or more of hydroxypropyl methylcellulose, hydroxypropylcellulose, polyvinyl pyrrolidone, methacrylates, and the like.

Misoprostol tablets may be prepared by blending misoprostol-polymer dispersion with other pharmaceutically acceptable excipients and compressing the blend into tablets.

The polymer in the misoprostol-polymer dispersion may be one or more of hydroxypropyl methylcellulose, hydroxypropylcellulose, polyvinylpyrrolidone, and the like.

In still another embodiment, diclofenac and misoprostol dosage form may be prepared by mixing coated minitablets of diclofenac or a salt thereof with beads of misoprostol or a salt thereof along with other pharmaceutically acceptable excipients and converting the final blend into a suitable dosage form.

Suitable dosage form may be in the form of a tablet, a caplet, a capsule, disc or any other dosage form for oral administration. The tablet dosage form may be coated.

The coated minitablets of diclofenac or a salt thereof may be prepared by mixing diclofenac or a salt thereof with other pharmaceutically acceptable excipients to form a blend; compressing the blend into minitablets; coating the minitablets of diclofenac or a salt thereof with a pharmaceutically acceptable seal coat polymer; enteric-coating the seal coated minitablets of diclofenac or a salt thereof with a pharmaceutically acceptable enteric coat polymer; and optionally, coating the enteric coated minitablets of diclofenac or a salt thereof with polyethylene glycol.

The beads of misoprostol or a salt thereof may be prepared by coating inert spherical beads with an alcoholic solution of misoprostol-polymer dispersion.

The alcohol used in the preparation of the alcoholic solution of misoprostol-polymer dispersion may be one or more of methanol, ethanol, propanol, isopropyl alcohol, and the like.

Suitable dosage form may be in the form of a tablet, a caplet, a capsule, disc or any other dosage form for oral administration. The tablet dosage form may be coated.

Suitable pharmaceutically acceptable seal coat polymers may include one or more of hydroxypropyl methylcellulose, hydroxypropyl cellulose and other suitable cellulose ethers.

Suitable pharmaceutically acceptable enteric coating polymers may include one or more of methacrylic acid/methyl methacrylate copolymers such as Eudragits or cellulose derivatives such as carboxymethyl cellulose, cellulose acetate phthalate, hydroxypropylmethyl cellulose phthalate, and other suitable polymers.

The dosage forms as described herein may include other pharmaceutically acceptable excipients. Examples of other pharmaceutically acceptable as used herein include binders, fillers, lubricants, disintegrants, glidants, and the like.

Suitable binders may include one or more of povidone, starch, stearic acid, gums, hydroxypropylmethyl cellulose, and the like.

Suitable fillers may include one or more of microcrystalline cellulose, lactose, mannitol, calcium phosphate, calcium sulfate, kaolin, dry starch, powdered sugar, and the like.

Suitable lubricants may include one or more of magnesium stearate, zinc stearate, calcium stearate, stearic acid, sodium stearyl fumarate, hydrogenated vegetable oil, and the like.

Suitable glidants may include one or more of colloidal silicon dioxide, talc or cornstarch, and the like.

Suitable disintegrants may include one or more of starch, croscarmellose sodium, crospovidone, sodium starch glycolate, and the like.

The present invention is further illustrated by the following examples which are provided merely to be exemplary of the invention and do not limit the scope of the invention. Certain modifications and equivalents will be apparent to those skilled in the art and are intended to be included within the scope of the present invention.

### EXAMPLE 1

TABLE 1

| No | Ingredients | % Composition |
|---|---|---|
| Misoprostol tablet | | |
| | Misoprostol:hypromellose | |
| | (1:100) | |
| 1 | Misoprostol | 1.01 to 2.0 |
| 2 | Hypromellose | 10 to 99 |
| 3 | Crospovidone | 1 to 10 |
| 4 | Colloidal silicon dioxide | 1.1 to 10 |
| 5 | Microcrystalline cellulose | 10 to 90 |
| 6 | Hydrogenated castor oil | 1.1 to 2.0 |
| Diclofenac sodium enteric | | |
| coated beads | | |
| | Beads | |
| 7 | Microcrystalline cellulose | 30 to 95 |
| | Drug layering | |
| 8 | Diclofenac sodium | 5 to 70 |
| 9 | Hypromellose | 3 to 25 |
| 10 | Polyethylene glycol | 1.15 to 2.5 |
| 11 | Purified water | q.s. |
| | Seal coating | |
| 12 | Hypromellose + PEG 400 | 1 to 5 |
| 13 | Purified water | q.s. |
| | Enteric coating | |
| 14 | Methacrylic acid copolymer | 8 to 25 |
| | suspension | |
| | (Methacrylic acid | |
| | copolymer, sodium | |
| | hydroxide, Talc, triethyl | |
| | citrate, purified water) | |
| | Blend for beads | |
| | compression | |
| 15 | PEG 6000 | 1.5 to 5 |
| 16 | Microcrystalline cellulose | 10 to 80 |
| 17 | Sodium starch glycolate | 1 to 10 |
| 18 | Hydrogenated castor oil | 1.1 to 2.0 |

Procedure: Misoprostol-hypromellose dispersion was mixed with microcrystalline cellulose, crospovidone, and colloidal silicon dioxide in a double cone blender. The above blend was lubricated with pre-sifted hydrogenated castor oil in a double cone blender and compressed into tablets using a suitable tooling.

Diclofenac sodium suspension was prepared in water along with hypromellose and polyethylene glycol under stirring. Microcrystalline cellulose beads were coated with diclofenac sodium suspension in a fluidized bed processor. The drug loaded beads thus obtained were seal coated with hypromellose and polyethylene glycol 400 solution. The seal coated drug loaded beads were coated with enteric polymer suspension prepared by mixing methacrylic acid polymer, sodium hydroxide, talc, triethyl citrate in water. The enteric-coated beads were lubricated with polyethylene glycol 6000 in a double cone blender and further mixed with microcrystalline cellulose, sodium starch glycolate. The above blend was lubricated with hydrogenated vegetable oil.

The diclofenac loaded enteric-coated beads blend was compressed along with misoprostol tablet such that misoprostol tablet was completely covered by diclofenac bead blend to form a tablet in a tablet dosage form using a suitable tooling. Finally, the tablet in a tablet was further coated with an aqueous dispersion of Opadry.

### EXAMPLE 2

TABLE 2

| No | Ingredients | % Composition |
|---|---|---|
| Misoprostol beads | | |
| 1 | Microcrystalline cellulose | 15-95 |
| | Misoprostol:hypromellose | |
| | (1:100) | |
| 2 | Misoprostol | 1.01-2.0 |
| 3 | Hypromellose | 10-99 |
| 4 | Isopropyl alcohol | q.s. |
| Diclofenac sodium | | |
| minitablets | | |
| | Drug layering | |
| 5 | Diclofenac sodium | 5-80 |
| 6 | Lactose | 10-90 |
| 7 | Sodium starch glycolate | 1-10 |
| 8 | Magnesium stearate | 1.1-3 |
| | Seal coating | |
| 9 | Hypromellose + PEG 400 | 1-5 |
| 10 | Purified water | q.s. |
| | Enteric coating | |
| 11 | Methacrylic acid copolymer | 8-25 |
| | suspension | |
| | (Methacrylic acid | |
| | copolymer, sodium | |
| | hydroxide, Talc, triethyl | |
| | citrate, purified water) | |
| 12 | PEG coating (PEG 6000, | 1-10 |
| | isopropyl alcohol) | |
| | Final Blend | |
| | Coated Diclofenac sodium | |
| | minitabs | |
| | Misoprostol beads | |
| 13 | Microcrystalline cellulose | 10-90 |
| 14 | Sodium starch glycolate | 1-10 |
| 15 | Colloidal silicon dioxide | 1-5 |
| 16 | Hydrogenated castor oil | 1.1 to 3.0 |

Procedure: Diclofenac sodium was mixed with lactose, sodium starch glycolate and lubricated with magnesium stearate. The lubricated blend was compressed into minitablets having weight between 10 mg to 50 mg. The diclofenac sodium minitablets were seal coated with hypromellose and polyethylene glycol 400 solution. The seal coated minitablets were enteric coated with coated with enteric polymer suspension prepared by mixing methacrylic acid polymer, sodium hydroxide, talc, triethyl citrate in water. The enteric-coated diclofenac sodium minitablets were further coated with polyethylene glycol 6000 solution prepared in isopropyl alcohol.

Misoprostol-hypromellose dispersion was dissolved in isopropyl alcohol and coated on microcrystalline cellulose beads in a fluidized bed processor.

The coated diclofenac sodium minitablets were mixed with misoprostol beads along with microcrystalline cellulose, sodium starch glycolate, colloidal silicon dioxide and lubricated with hydrogenated castor oil. The final blend was compressed into tablets using a suitable tooling. The compressed tablets were further coated with an aqueous dispersion of Opadry.

### EXAMPLE 3

TABLE 3

| No | Ingredients | % Composition |
|---|---|---|
| Misoprostol tablet | | |
| | Misoprostol:hypromellose | |
| | (1:100) | |
| 1 | Misoprostol | 1.01 to 2.0 |
| 2 | Hypromellose | 10 to 99 |
| 3 | Crospovidone | 1 to 10 |
| 4 | Colloidal silicon dioxide | 1.1 to 10 |
| 5 | Microcrystalline cellulose | 10 to 90 |
| 6 | Hydrogenated castor oil | 1.1 to 2.0 |
| Diclofenac sodium enteric | | |
| coated beads | | |
| | Beads | |
| 1 | Microcrystalline cellulose | 30 to 95 |
| | Drug layering | |
| 2 | Diclofenac sodium | 5 to 70 |
| 3 | Hypromellose | 3 to 25 |
| 4 | Polyethylene glycol | 1.15 to 2.5 |
| 5 | Purified water | q.s. |
| | Seal coating | |
| 6 | Hypromellose + PEG 400 | 1 to 5 |
| 7 | Purified water | q.s. |
| | Enteric coating | |
| 8 | Methacrylic acid copolymer | 8 to 25 |
| | suspension | |
| | (Methacrylic acid | |
| | copolymer, sodium | |
| | hydroxide, Talc, triethyl | |
| | citrate, purified water) | |
| | Blend for beads | |
| | compression | |
| 9 | PEG 6000 | 1.5 to 5 |
| 10 | Microcrystalline cellulose | 10 to 80 |
| 11 | Sodium starch glycolate | 1 to 10 |
| 12 | Hydrogenated castor oil | 1.1 to 2.0 |

Procedure: Misoprostol-hypromellose dispersion was mixed with microcrystalline cellulose, crospovidone, and colloidal silicon dioxide in a double cone blender. The above blend was lubricated with pre-sifted hydrogenated castor oil in a double cone blender and compressed into tablets using a suitable tooling.

Diclofenac sodium suspension was prepared in water along with hypromellose and polyethylene glycol under stirring. Microcrystalline cellulose beads were coated with the diclofenac sodium suspension in a fluidized bed processor. The drug loaded beads thus obtained were seal coated with hypromellose and polyethylene glycol 400 solution. The seal coated drug loaded beads were coated with enteric polymer suspension prepared by mixing methacrylic acid polymer, sodium hydroxide, talc, triethyl citrate in water. The enteric-coated beads were lubricated with polyethylene glycol 6000 in a double cone blender and further mixed with microcrystalline cellulose, sodium starch glycolate. The above blend was lubricated with hydrogenated vegetable oil.

The diclofenac loaded enteric-coated beads blend was compressed along with misoprostol tablet in such a way that misoprostol tablet was tilted on the one side of diclofenac tablet surface, such that it looks like a pillow at the center.

Finally, the pillow tablet was further coated with an aqueous dispersion of Opadry.

### EXAMPLE 4

TABLE 4

| No | Ingredients | % Composition |
|---|---|---|
| Misoprostol tablet | | |
| | Misoprostol:hypromellose | |
| | (1:100) | |
| 1 | Misoprostol | 1.01 to 2.0 |
| 2 | Hypromellose | 10 to 99 |
| 3 | Crospovidone | 1 to 10 |
| 4 | Colloidal silicon dioxide | 1.1 to 10 |
| 5 | Microcrystalline cellulose | 10 to 90 |
| 6 | Hydrogenated castor oil | 1.1 to 2.0 |
| Diclofenac sodium enteric | | |
| coated beads | | |
| | Beads | |
| 7 | Microcrystalline cellulose | 30 to 95 |
| | Drug layering | |
| 8 | Diclofenac sodium | 5 to 70 |
| 9 | Hypromellose | 3 to 25 |
| 10 | Polyethylene glycol | 1.15 to 2.5 |
| 11 | Purified water | q.s. |
| | Seal coating | |
| 12 | Hypromellose + PEG 400 | 1 to 5 |
| 13 | Purified water | q.s. |
| | Enteric coating | |
| 14 | Methacrylic acid copolymer | 8 to 25 |
| | suspension | |
| | (Methacrylic acid | |
| | copolymer, sodium | |
| | hydroxide, Talc, triethyl | |
| | citrate, purified water) | |
| | Blend for beads | |
| | compression | |
| 15 | PEG 6000 | 1.5 to 5 |
| 16 | Microcrystalline cellulose | 10 to 80 |
| 17 | Sodium starch glycolate | 1 to 10 |
| 18 | Hydrogenated castor oil | 1.1 to 2.0 |

Procedure: Misoprostol-hypromellose dispersion was mixed with microcrystalline cellulose, crospovidone, and colloidal silicon dioxide in a double cone blender. The above blend was lubricated with pre-sifted hydrogenated castor oil in a double cone blender and compressed into tablets using a suitable tooling.

The diclofenac sodium suspension was prepared in water along with hypromellose and polyethylene glycol under stirring. Microcrystalline cellulose beads were coated with the diclofenac sodium suspension in a fluidized bed processor. The drug loaded beads thus obtained were seal coated with hypromellose and polyethylene glycol 400 solution. The seal coated drug loaded beads were coated with enteric polymer suspension prepared by mixing methacrylic acid polymer, sodium hydroxide, talc, triethyl citrate in water. The enteric-coated beads were lubricated with polyethylene glycol 6000 in a double cone blender and further mixed with microcrystalline cellulose, sodium starch glycolate. The above blend was lubricated with hydrogenated vegetable oil.

The diclofenac loaded enteric-coated beads blend was compressed along with misoprostol tablet in such a way that misoprostol tablet was inlayed on the one side of diclofenac tablet surface.

The inlay tablet was further coated with an aqueous dispersion of Opadry.

### EXAMPLE 5

TABLE 5

| No | Ingredients | % Composition |
|---|---|---|
| Misoprostol coating | | |
| | Misoprostol:hypromellose | |
| | (1:100) | |
| 1 | Misoprostol | 1.01 to 2.0 |
| 2 | Hypromellose | 10 to 99 |
| Diclofenac sodium enteric | | |
| coated beads | | |
| | Beads | |
| 1 | Microcrystalline cellulose | 30 to 95 |
| | Drug layering | |
| 2 | Diclofenac sodium | 5 to 70 |
| 3 | Hypromellose | 3 to 25 |
| 4 | Polyethylene glycol | 1.15 to 2.5 |
| 5 | Purified water | q.s. |
| | Seal coating | |
| 6 | Hypromellose + PEG 400 | 1 to 5 |
| 7 | Purified water | |
| | Enteric coating | |
| 8 | Methacrylic acid copolymer | 8 to 25 |
| | suspension | |
| | (Methacrylic acid | |
| | copolymer, sodium | |
| | hydroxide, Talc, triethyl | |
| | citrate, purified water) | |
| | Blend for beads | |
| | compression | |
| 9 | PEG 6000 | 1.5 to 5 |
| 10 | Microcrystalline cellulose | 10 to 80 |
| 11 | Sodium starch glycolate | 1 to 10 |
| 12 | Hydrogenated castor oil | 1.1 to 2.0 |

Procedure: Diclofenac sodium suspension was prepared in water along with hypromellose and polyethylene glycol under stirring. Microcrystalline cellulose beads were coated with the diclofenac sodium suspension in a fluidized bed processor. The drug loaded beads thus obtained were seal coated with hypromellose and polyethylene glycol 400 solution. The seal coated drug loaded beads were coated with enteric polymer suspension prepared by mixing methacrylic acid polymer, sodium hydroxide, talc, triethyl citrate in water. The enteric-coated beads were lubricated with polyethylene glycol 6000 in a double cone blender and further mixed with microcrystalline cellulose, sodium starch glycolate. The above blend was lubricated with hydrogenated vegetable oil.

The diclofenac loaded enteric-coated beads blend was divided into two parts in such a way that first part contained 90% of the enteric-coated beads of diclofenac and other second part contained the remaining 10% of the enteric-coated beads of diclofenac. The first part containing 90% of the enteric-coated beads of diclofenac was blended with misoprostol-hypromellose dispersion in a double cone blender. This blend of enteric-coated beads of diclofenac coated with misoprostol-hypromellose was compressed with the second part containing remaining 10% diclofenac loaded enteric-coated beads into bilayered tablets using a suitable tooling. Finally, the tablet was further coated with an aqueous dispersion of Opadry.

While the invention has been described in terms of its specific embodiments, certain modifications and equivalents will be apparent to those skilled in the art and are intended to be included within the scope of the invention.

1. A tablet in a tablet dosage form comprising:
(a) an inner tablet comprising misoprostol or a salt thereof and optionally other pharmaceutically acceptable excipients; and
(b) an outer tablet comprising coated beads of diclofenac or a salt thereof and optionally other pharmaceutically acceptable excipients.

2. The dosage form of claim 1, wherein the coated beads of diclofenac or a salt thereof are prepared by a process comprising;
(a) coating inert spherical beads with a suspension of diclofenac or a salt thereof;
(b) overcoating the diclofenac loaded beads of step a) with a pharmaceutically acceptable seal coat polymer;
(c) enteric-coating the seal coated diclofenac beads of step b) with a pharmaceutically acceptable enteric coat polymer; and
(d) optionally, mixing the enteric-coated beads of diclofenac or a salt thereof with polyethylene glycol and optionally with other pharmaceutically acceptable excipients.

3. The dosage form of claim 2, wherein the pharmaceutically acceptable seal coat polymer comprises one or more of hydroxypropyl methylcellulose, hydroxypropyl cellulose and cellulose ethers.

4. The dosage form of claim 2, wherein the pharmaceutically acceptable enteric coating polymer comprises one or more of methacrylic acid/methyl methacrylate copolymers, cellulose acetate phthalate, hydroxypropylmethyl cellulose phthalate, hydroxypropylmethyl cellulose acetate succinate, polyvinyl acetate phthalate and cellulose acetate trimellitate.

5. The dosage form of claim 1, wherein the dosage form exhibits a dissolution profile such that within first 2 hours less than 2% of diclofenac or a salt thereof is released, when the release rate is measured in Apparatus 2 (USP, Dissolution, paddle, 50 rpm) using 900 ml of 0.1 N HCl at 37° C.±0.5° C. and within first 30 minutes more than 80% of diclofenac or a salt thereof is released, when the release rate is measured in Apparatus 2 (USP, Dissolution, paddle, 50 rpm) using 1000 ml of pH 6.8 phosphate buffer at 37° C.±0.5° C.

6. A dosage form comprising coated minitablets of diclofenac or a salt thereof optionally, with other pharmaceutically acceptable excipients and beads of misoprostol or a salt thereof optionally, with other pharmaceutically acceptable excipients.

7. The dosage form of claim 6, wherein the coated minitablets of diclofenac or a salt thereof are prepared by a process comprising:
(a) mixing diclofenac or a salt thereof with other pharmaceutically acceptable excipients to form a blend;
(b) compressing the blend into minitablets;
(c) coating the minitablets of diclofenac or a salt thereof with a pharmaceutically acceptable seal coat polymer;
(d) enteric-coating the seal coated minitablets of diclofenac or a salt thereof with a pharmaceutically acceptable enteric coat polymer; and
(e) optionally, coating the enteric coated minitablets of diclofenac or a salt thereof with polyethylene glycol.

8. The dosage form of claim 7, wherein the pharmaceutically acceptable seal coat polymer comprises one or more of hydroxypropyl methylcellulose, hydroxypropyl cellulose and cellulose ethers.

9. The dosage form of claim 7, wherein the pharmaceutically acceptable enteric coating polymer comprises one or more of methacrylic acid/methyl methacrylate copolymers, cellulose acetate phthalate, hydroxypropylmethyl cellulose phthalate, hydroxypropylmethyl cellulose acetate succinate, polyvinyl acetate phthalate and cellulose acetate trimellitate.

10. The dosage form of claim 6, wherein the dosage form exhibits a dissolution profile such that within first 2 hours less than 2% of diclofenac or a salt thereof is released, when the release rate is measured in Apparatus 2 (USP, Dissolution, paddle, 50 rpm) using 900 ml of 0.1 N HCl at 37° C.±0.5° C. and within first 30 minutes more than 80% of diclofenac or a salt thereof is released, when the release rate is measured in Apparatus 2 (USP, Dissolution, paddle, 50 rpm) using 1000 ml of pH 6.8 phosphate buffer at 37° C.±0.5° C.

11. A pillow tablet dosage form comprising: an inner pillowed tablet comprising:
(a) misoprostol or a salt thereof and optionally other pharmaceutically acceptable excipients; and
(b) an outer tablet comprising coated beads of diclofenac or a salt thereof and optionally other pharmaceutically acceptable excipients.

12. The dosage form of claim 11, wherein the coated beads of diclofenac or a salt thereof are prepared by a process comprising:
(a) coating inert spherical beads with a suspension of diclofenac or a salt thereof;
(b) overcoating the diclofenac loaded beads of step a) with a pharmaceutically acceptable seal coat polymer;
(c) enteric-coating the seal coated diclofenac beads of step b) with a pharmaceutically acceptable enteric polymer; and
(d) optionally, mixing the enteric-coated beads of diclofenac or a salt thereof of step c) with polyethylene glycol and other pharmaceutically acceptable excipients.

13. The dosage form of claim 12, wherein the pharmaceutically acceptable seal coat polymer comprises one or more of hydroxypropyl methylcellulose, hydroxypropyl cellulose and cellulose ethers.

14. The dosage form of claim 12, wherein the pharmaceutically acceptable enteric coating polymer comprises one or more of methacrylic acid/methyl methacrylate copolymers, cellulose acetate phthalate, hydroxypropylmethyl cellulose phthalate, hydroxypropylmethyl cellulose acetate succinate, polyvinyl acetate phthalate and cellulose acetate trimellitate.

15. The dosage form of claim 11, wherein the dosage form exhibits a dissolution profile such that within first 2 hours less than 2% of diclofenac or a salt thereof is released, when the release rate is measured in Apparatus 2 (USP, Dissolution, paddle, 50 rpm) using 900 ml of 0.1 N HCl at 37° C.±0.5° C. and within first 30 minutes more than 80% of diclofenac or a salt thereof is released, when the release rate is measured in Apparatus 2 (USP, Dissolution, paddle, 50 rpm) using 1000 ml of pH 6.8 phosphate buffer at 37° C.±0.5° C.

16. An inlay tablet dosage form comprising:
(a) an inner inlayed tablet comprising misoprostol or a salt thereof and optionally other pharmaceutically acceptable excipients; and
(b) an outer tablet comprising coated beads of diclofenac or a salt thereof and optionally other pharmaceutically acceptable excipients.

17. The dosage form of claim 16, wherein the coated beads of diclofenac or a salt thereof are prepared by a process comprising:
(a) coating inert spherical beads with a suspension of diclofenac or a salt thereof;
(b) overcoating the diclofenac loaded beads of step a) with a pharmaceutically acceptable seal coat polymer;
(c) enteric-coating the seal coated diclofenac beads of step b) with a pharmaceutically acceptable enteric coat polymer; and
(d) optionally, mixing the enteric-coated beads of diclofenac or a salt thereof of step c) with polyethylene glycol and other pharmaceutically acceptable excipients.

18. The dosage form of claim 17, wherein the pharmaceutically acceptable seal coat polymer comprises one or more of hydroxypropyl methylcellulose, hydroxypropyl cellulose and cellulose ethers.

19. The dosage form of claim 17, wherein the pharmaceutically acceptable enteric coating polymer comprises one or more of methacrylic acid/methyl methacrylate copolymers, cellulose acetate phthalate, hydroxypropylmethyl cellulose phthalate, hydroxypropylmethyl cellulose acetate succinate, polyvinyl acetate phthalate and cellulose acetate trimellitate.

20. The dosage form of claim 16, wherein the dosage form exhibits a dissolution profile such that within first 2 hours less than 2% of diclofenac or a salt thereof is released, when the release rate is measured in Apparatus 2 (USP, Dissolution, paddle, 50 rpm) using 900 ml of 0.1N HCl at 37° C.±0.5° C. and within first 30 minutes more than 80% of diclofenac or salt thereof is released, when the release rate is measured in Apparatus 2 (USP, Dissolution, paddle, 50 rpm) using 1000 ml of pH 6.8 phosphate buffer at 37° C.±0.5° C.

21. A dosage form comprising coated beads of diclofenac or a salt thereof optionally, with other pharmaceutically acceptable excipients and a coating comprising misoprostol or a salt thereof optionally, with other pharmaceutical acceptable excipients, characterized in that said misoprostol coating covers not more than 90% of the coated beads of diclofenac or a salt thereof.

22. The dosage form of claim 21, wherein the coated beads of diclofenac or a salt thereof are prepared by a process comprising:
(a) coating inert spherical beads with a suspension of diclofenac or a salt thereof;
(b) overcoating the diclofenac loaded beads of step a) with a pharmaceutically acceptable seal coat polymer;
(c) enteric-coating the seal coated diclofenac beads of step b) with a pharmaceutically acceptable enteric coat polymer; and
(d) optionally mixing the enteric-coated beads of diclofenac or a salt thereof of step c) are mixed with polyethylene glycol along with other pharmaceutically Acceptable excipients.

23. The dosage form of claim 22, wherein the pharmaceutically acceptable seal coat polymer comprises one or more of hydroxypropyl methylcellulose, hydroxypropyl cellulose and cellulose ethers.

24. The dosage form of claim 22, wherein the pharmaceutically acceptable enteric coating polymer comprises one or more of methacrylic acid/methyl methacrylate copolymers, cellulose acetate phthalate, hydroxypropylmethyl cellulose phthalate, hydroxypropylmethyl cellulose acetate succinate, polyvinyl acetate phthalate and cellulose acetate trimellitate.

25. The dosage form of claim 21, wherein the dosage form exhibits a dissolution profile such that within first 2 hours less than 2% of diclofenac or a salt thereof is released, when the release rate is measured in Apparatus 2 (USP, Dissolution, paddle, 50 rpm) using 1000 ml of 0.1N HCl at 37° C.±0.5° C. and within first 30 minutes more than 80% of diclofenac or a salt thereof is released, when the release rate is measured in Apparatus 2 (USP, Dissolution, paddle, 50 rpm) using 1000 ml of pH 6.8 phosphate buffer at 37° C.±0.5° C.
